(19) United States
(12) Patent Application Publication
(10) Pub. No.: US 2014/0243867 A1
(43) Pub. Date: Aug. 28, 2014

(54) SURGICALLY INVASIVE POINTED ARTICLES FOR SKIN PENETRATION AND METHODS FOR THEIR PREPARATION

(71) Applicant: Chee Yen Lim, Singapore (SG)

(72) Inventor: Chee Yen Lim, Singapore (SG)

(21) Appl. No.: 14/351,894

(22) PCT Filed: Nov. 2, 2011
(86) PCT No.: PCT/SG2011/000391

§ 371 Date: Apr. 15, 2014

(51) Int. Cl. A61B 5/151; B29C 45/14

(52) U.S. Cl. CPC A61B 5/15134; B29C 45/14
USPC 606/181; 264/250

(57) ABSTRACT

A surgically invasive pointed article for skin penetration and its fabrication method are presented. The present invention comprises a protruding sharp end portion and a body portion, the protruding sharp end portion comprising at least one penetration tip that can be disabled after use. The tip disablement after use is an effective safety measure in eradicating accidental injuries and re-use. The tip disablement can be carried out manually by the user or automatically if the article is used with a custom-made tip-disabling applicator. The skin penetration device can be used as a lancet for blood sampling, a needle for immunization and skin allergy testing, or a skin breaching device for transdermal drug delivery. The second aspect of the invention is a method for preparing the surgically invasive pointed article for skin penetration. The preparation method includes injection mould the articles into at least two body segments, the first body segment comprising at least one protruding end for skin penetration, and the second body segment being hollow forming a handle for handling purpose or an adaptor for fitting into an applicator. The third aspect of the invention is an over-moulding process for packaging purposes, wherein the articles moulded with a first material mentioned above are over-moulded with a second softer and cheaper material to form various additional functional parts.

### BACKGROUND OF THE INVENTION

1. Field of the Invention

This invention relates generally to a surgically invasive medical device, and more particularly to a pointed article for skin penetration whose tip can be disabled after use to prevent accidental injuries and re-use.

2. Background of the Invention

### Skin Penetration for Medical Applications

Skin penetration is insertion of at least a pointed article into the skin with depths ranging from tens of microns to less than three millimetres. Depending on the purpose, there are several methods and devices currently available for skin penetration. For transdermal drug delivery purpose, small cluster of needles (microneedles) are inserted into the skin to puncture the outer layer of skin which is otherwise a barrier for drug delivery into the skin. The term ‘drugs’ is a general referred to medicinal substance such as hormones, drugs, vaccines etc. Such microneedles are sometimes inserted into the skin by bare hand or a hand held device to extract bodily fluids such as plasma etc. for analysis. Most of these devices and methods are still in clinical trial phase and are not available on the market. These devices are normally made in sophisticated processes. In addition, they require extra packaging effort in keeping them sterile till point of use and extra care for disposal to prevent accidental injuries or re-use.

For blood sampling purpose, the pointed article (better known as blood lancets) is used with a spring-operated applicator which inserts the blood lancet into the skin and retracts it at a very high speed. Since the speed is very high (often more than 1,000 mm/sec), the duration of the blood lancet staying in the skin is very brief (often a few milliseconds), so the discomfort or pain incurred is minimal. The applicator's distal end which contacts the skin has an opening for the lancet to penetrate the skin. The opening also serves an important role in fixing the skin such that certain rigidity and surface tension are reached for optimal skin penetration. Without the fixation of the skin, the skin penetration result will be inconsistent and much more painful. The blood lancets are made by over-moulding a stainless steel needle with inexpensive polymer such as polyethylene or polypropylene to form a handle. A cap, which conditions till point of use. Although this manufacturing method requires no extra packaging effort, there are still problems in handling and making the stainless steel needle. Moreover, the device comprising stainless steel and polymer is difficult to recycle as the stainless steel (1200 degree Celsius) has much higher melting temperature to polymers (<350 degree Celsius). In addition, the stainless steel needle imposes an injury threat to the medical personnel who handle the disposal of the lancets. The cap is not an adequate measure in preventing accidental injuries or re-use because it may not be capped after use or may be removed accidentally or intentionally.

### Closest Prior Arts

It is known from U.S. Pat. No. 3,358,689 that stainless steel lancets can be over-moulded such that the needle is covered fully by a plastic material, a portion of which can be removed to expose the tip at point of use. In this case, the making of the needle constitute most of the cost as stainless steel wire is expensive and multiple steps are involved in cutting, grinding, and polishing the wire into needles before over-moulding is done. This patent '689 regarding an “integral lancet and package” was related to over-moulding of a stainless steel needle using a polymer material. The stainless steel needle is over-moulded with polymer to form a plastic body for handling purpose and a cap to eliminate packaging. With the over-moulded cap, the lancets can be sterilized and kept sterile till point of use. The over-moulding process on stainless needles does not provide make use of polymers which can be moulded to save manufacturing and material costs.

Yet another prior art, U.S. Pat. No. 4,545,376, was related to a blood lancet reported a flat lancet with a protective yoke, which was made of plastic material. The patented article was made by injection moulding and the tip and the body was made of same material (polymer). However, the yokes that protected the needles during handling were not capable of preserving the sterile conditions of the tip as the yoke did not encapsulate the tip fully. This invention also did not disclose any safety measure (such as tip disablement).

Another prior art, U.S. Pat. No. 5,250,066 was related to plastic lancets made of shear-thinned thermoplastic polymer selected from the group consisting of a liquid crystal polymer and modified acrylic polymer. The invention proposed lancets which were made fully of one plastic material via shear-thinning during the injection moulding process and did not include over-moulding process of a second plastic material.

Accordingly, there is a long-felt need in having a skin penetration device that eliminates accidental injuries and re-use, which is made and packaged cost efficiently. The present invention is directed to fulfilling the need.

### Solutions to Exiting Problems

1. The current skin penetration devices have either no or inadequate safety measure in preventing accidental injuries and re-use. The use of cap to contain the sharp tip in blood lancets does not thoroughly eliminate the risk of accidental injuries or re-use, it is just reducing the risk. The use of safety lancets is an effective solution but is too expensive for disposable items. Some skin penetration devices are made of inexpensive materials and methods, but all of these devices require extra packaging step to conserve the sterile conditions till point of use.

2. The current pointed articles for skin penetration are not cost efficient in terms of manufacturing and packaging, since they are disposable items, they should be made in a simple manufacturing process requiring no extra packaging steps so that the cost can be brought down significantly. Costs incurred for grinding stainless steel needles are eliminated.

### SUMMARY OF THE INVENTION

Skin penetration is common in medical practice. The two primary objectives for skin penetration are for (i) delivering medicinal substance to the skin or (ii) extracting bodily fluid for analysis. For the first objective, skin penetration is done in immunization, skin allergy testing, transdermal/intradermal drug delivery, etc. The sharp tips involved are normally more than one, although a single tip may be adequate depending on the delivery volume. For the second objective, skin penetration is done in blood sampling in which a small sample of blood is obtained for further analysis.

The skin penetration devices on the market include vaccination and skin testing needles, microneedles and lancets. For vaccination and skin testing needles, the user normally inserts the needle by bare hand. On the other hand, microneedles are applied to skin by a user using bare hand or a spring-operated applicator. Similarly, almost all lancets are used with a spring-operated applicator. Where possible, a spring-operated applicator is usually preferred over bare hand as the applicator provides consistent penetration speed and depth, which is important in getting consistent results and reducing the pain level.

Most of the skin penetration devices mentioned above do not have any or adequate disposal measure to ensure safety or prevent re-use. Some safety measure such as a cap does not guarantee absolute safety as the cap can be coming off or the user may unintentionally omit the step. The safety cap also fails to prevent re-use, which is a major cause of cross infection and spread of disease.

Since these skin penetration devices are disposable after use, their price per unit should be adequately economical to discourage re-use. The current way of making and packaging these devices impose high barrier in further reducing their costs. For example, these skin penetration devices are packaged in a rigid container to protect the device and to conserve the sterile conditions till point of use. The rigid package and the packaging job increase the cost.

Understanding the shortcomings of the prior arts, the present invention is developed to provide several advantages over the prior arts. The present invention is targeted to provide a plastic needle that can be moulded from a suitable polymer to provide the strength required for skin penetration, while the rest of the pointed article can be over-moulded with an inexpensive polymer. This method presents a cost advantage over the current stainless steel lancet when taking into consideration the material cost (by weight) and the number of steps involved in making stainless steel needles. The present invention is further targeted to provide a safety feature of tip disablement after use for preventing accidental injuries and re-use, which is a superior feature over the prior art. The present invention is also targeted to include an over-moulding process which is capable of incorporating a cap and a body from a second polymer to conserve the sterile conditions of the tip till point of use and to save material cost as only a small quantity of high performance material is needed to mould the needle structure.

The first aspect of the present invention is to have a surgically invasive pointed article for skin penetration with a protruding sharp end portion and a body portion, the protruding sharp end portion comprising at least one penetration tip that can be disabled after use. The tip disablement after use is an effective safety measure in eradicating accidental injuries and re-use. The tip disablement can be carried out manually by the user or automatically if the article is used with a custom-made tip-disabling applicator. The skin penetration device can be used as a lancet for blood sampling, a needle for immunization and skin allergy testing, or a skin breaching device for transdermal drug delivery. The article is injection moulded as a single piece with materials hard enough to provide the required penetration force but soft enough to deform when compressed against a harder surface. The choice of material is dependent upon the size of the tips and the required penetration strength. Some suitable materials include metals such as zinc and stainless steel and bio-compatible acrylics, polycarbonate, polyetherimide, etc.

The second aspect of the invention is a method for preparing the surgically invasive pointed article for skin penetration. The preparation method includes injection mould the articles into at least two body segments, the first body segment comprising at least one protruding end for skin penetration, and the second body segment being hollow forming a handle for handling purpose or an adaptor for fitting into an applicator. The second body segment which is hollow saves some material and allows the injection moulded articles to be stacked on one another to protect the tips from damage and once sterilized, to conserve the sterile conditions till point of use.

The third aspect of the invention is a method of packaging the injection moulded articles. For example, the articles moulded with a first material mentioned above are over-moulded with a second softer and cheaper material to form various additional functional parts. To further illustrate, a detachable cap, an adaptor or both can be formed using the second material. To over-mould a detachable cap, the articles can be partially over-moulded such that only the protruding sharp end portion is fully covered with a second material, which does not form chemical bonding with the first material after the over-moulding process. To over-mould an adaptor, the articles are over-moulded partially such that the protruding sharp end portion is left open. Similarly, the articles can be fully over-moulded by a second material which does not form chemical bonding with the first material after the over-moulding process to form a cap and an adaptor. The advantages are clear and significant: (i) the skin penetration article which require expensive moulding material can be made small to save material cost, while the rest of the parts can be made up by a second material which is softer and cheaper and does not form chemical bonding with the first material; (ii) the over-moulding process provides an effective form of packaging in that the sharp tips are isolated from the ambient so that they are protected from damage and once sterilized, their sterile conditions are kept till point of use, i.e. when the user pulls apart the cap.

As illustrated above, the present invention provides a surgically invasive pointed article for skin penetration whose penetration ability can be disabled after use by deforming the tips. The article's body can be made hollow so that they can be stacked on one another to isolate the tips from the ambient. Alternatively, the article is made by injection moulding using a first material, followed by another injection moulding process to over-mould a second material to isolate the article from the ambient to protect the tips from damage and once sterilized, to conserve the sterile conditions till point of use. The over-moulding process adds additional functional parts to the article, namely a detachable cap, an adaptor or both. The article can be used for applications such as vaccination or skin allergy testing, blood sampling and transdermal/intradermal drug delivery. The article has a first body segment of a protruding pointed end comprising at least one sharp tip and a second body segment for handling purpose or for over-moulding purpose.

### DESCRIPTION OF THE INVENTION

### BRIEF DESCRIPTION OF THE FIGURES

FIG. 1 illustrates a typical skin penetration device

FIG. 2 illustrates the modes of tip disablement

FIG. 3 illustrates the needle segment of the skin penetration device

FIG. 4 illustrates the body segment of the skin penetration device

FIG. 5 illustrates the over-moulding of the cap onto the skin penetration device

FIG. 6 illustrates the over-moulding of the cap and the body onto the skin penetration device

### DETAILED DESCRIPTION OF THE INVENTION

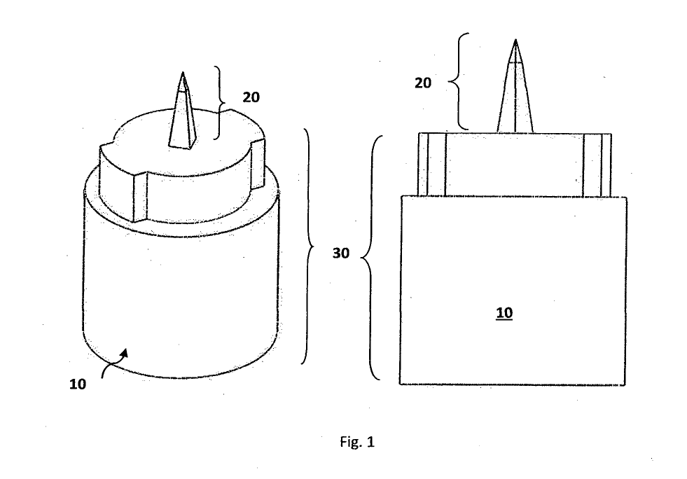

The preferred embodiment of the present invention involves a surgically invasive skin penetration device 10 comprising at least two body segments, as shown in FIG. 1. The first body segment 20 comprises a protruding pointed end for penetrating the skin and the second body segment 30 comprises a body portion which is extended from the first body segment 20 and which has a body shape and size suitable for handling or fitting into an applicator. The device 10 is preferably made in one piece using a suitable material such that the first body. segment 20 can be disabled from penetrating the skin after use. This act of disabling the penetration function of the device 10 can be achieved by deforming or removing the first body segment 20. For example, the used device 10 can be pressed against a hard surface, or hit or cut by a mechanism to remove the sharp tip thereby disabling the skin penetration function.

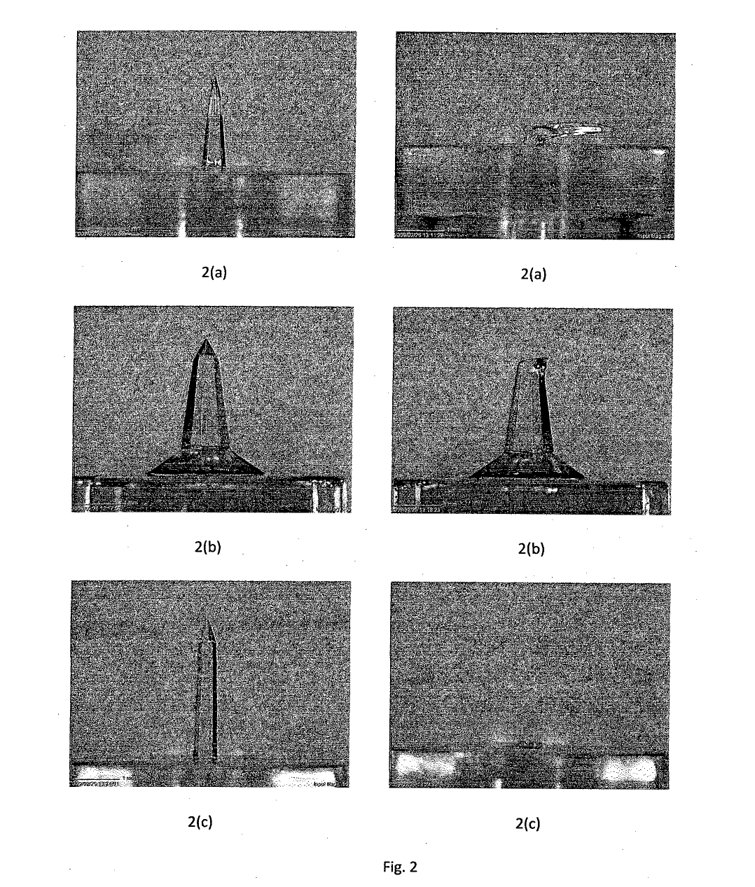

FIG. 2 shows an original and some disabled pointed ends of the first body segment 20. In one example as shown in FIG. 2(a), the entire first body segment 20 is bent substantially 90 degrees such that the sharp tip is in a ‘retired’ position within the end surface of the second body segment 30. In another example in FIG. 2(b), only the sharp end of the first body segment 20 is deformed physically to blunt the sharp end. In the third example in FIG. 2(c), the sharp end is removed by cutting it away. The tip disablement function in the device 10 presents a cost effective way to prevent accidental injuries and re-use. A preferred method for manufacturing the device 10 is by injection moulding. Materials used for injection moulding the device 10 should be bio-compatible and preferably gamma-ray sterible, among them are metals and polymers with sufficient impact and flexural strength, such as polycarbonate and polyetherimide.

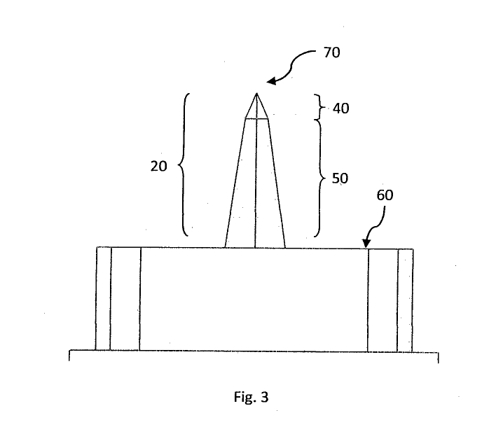

FIG. 3 shows the first body segment 20 of the present invention. The first body segment is now referred to as the ‘needle segment’ hereinafter. Preferably, the needle segment 20 has a total body length of 0.5 mm to 3 mm, and is generally tapered towards the distal end. The taper shape of the needle segment 20 is to allow a very sharp tip at the distal end for skin penetration and a larger body cross section to increase the structural strength and hardness of the needle segment 20. The geometry of the needle segment 20 can be of circular, triangular or polygonal shape so long as it can be conveniently made. In one example the geometry of the needle segment 20 is symmetrical for easy fabrication of mould, the preferred geometries include but not limited to round shape and rectangular shape. Although the device 10 may be of an arbitrary length, say 3 mm, usually only a portion of the length of the needle segment 20 is used to penetrate the skin. The typical skin penetration depth ranges from 0.05 mm to 2-3 mm for most skin penetration purposes and applications. To effectively increase the sharpness and the body strength of the needle segment, the needle segment can be made into two portions namely the needle body 40 and the needle tip 50. The needle body 40 comprises a long and slender body generally tapered from the base 60 to the distal end 70. The needle tip 50 can be made of a second taper extending from the tapered body, or can be bevelled such that the tip is at the side not centre to achieve the required sharpness. The length of the needle tip 50 is dependent on the penetration depth required, and is normally from 0.25 mm to 1.50 mm or more typically from 0.5 mm to 1 mm.

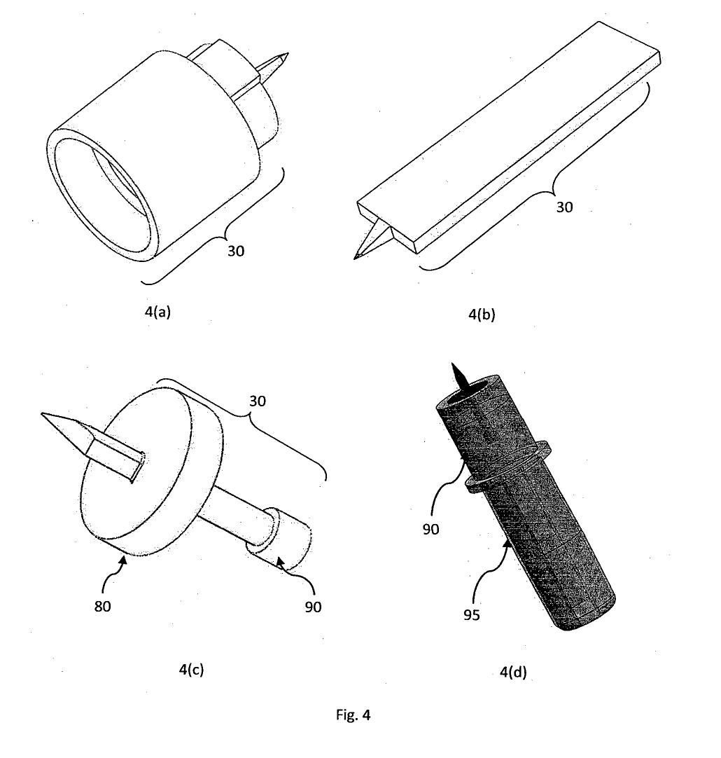

FIG. 4 shows a series of body shapes that the second body segment 30 may be injection moulded with a suitable material mentioned previously, hereinafter “the first material”. As shown in FIG. 4(a), the second body segment 30 can have a hollow body, or any arbitrary shape for handling or fitting purpose, or substantially similar to the first body segment 20 (i.e. a longer needle, not shown). FIG. 4(b) shows a second body segment 30 that is substantially flat and rectangular. Alternatively, FIG. 4(c) shows a second body segment 30 that includes at least a flange 80 for non-chemical contact with a second material during over-moulding process. As shown in FIG. 4(d), the anchor 90 can be disposed on the second body segment 30 so that the device 10 is fixed in the over-moulded body 95 when over-moulded by a second material. The anchor 90 can be a raised or indent features (groove or notch) which differ from the body segment 30 cross-sectional wise, the anchor 90 will be able to resist sliding of the body segment 30 due to difference in cross sectional areas. The over-moulded body 95 can take any form other than illustrated herein. Some of the suitable candidates for the second material are polyethylene, polypropylene, silicone or other similar polymers which have much lower melting temperature than the first material and which preferably do not form chemical bonding with the first material. The second material is also preferably much softer than the first material. Over-moulding of a second material can save material cost by making up the uncritical second body segment 30 with softer but much inexpensive material. Without the gripping features, the over-moulded body may be slip off the device 10 due to no chemical bonding between the materials.

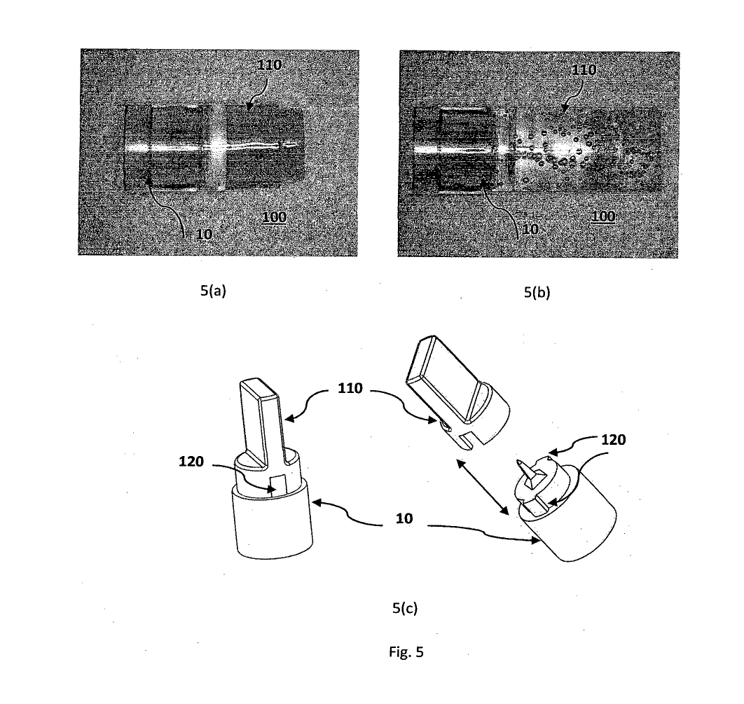

Another advantage of over-moulding using the second material is that a cap can be over-moulded to contain the needle segment 20 in FIG. 1 such that the sharpness and the sterile conditions can be preserved till point-of-use. FIG. 5 shows a preferred embodiment of a capped skin penetration device 100, which is made of an over-moulded cap 110 and an injection moulded skin penetration device 10. For example, FIG. 5(a) shows a capped skin penetration device 100 which comprises a skin penetration device 10 made of polycarbonate with melting temperature in the range of 265 degrees Celsius and an over-moulded a cap 110 using polyethylene with melting temperature in the range of 115 degrees Celsius. The shape of the cap 110 and the device 10 is not critical and it can take any convenient form other than shown in the present embodiments. FIG. 5(b) shows a capped skin penetration device 100 with RTV silicone material cap 110. The bubbles in the figure serve no other purpose than to contrast the transparent silicone. FIG. 5(c) shows schematically how the cap 110 is pulled off from the skin penetration device 10. At least one ear 120 is disposed on the skin penetration device to guide the movement of the cap while it is pulled off. The ear 120 prevents turning movement which may damage the needle.

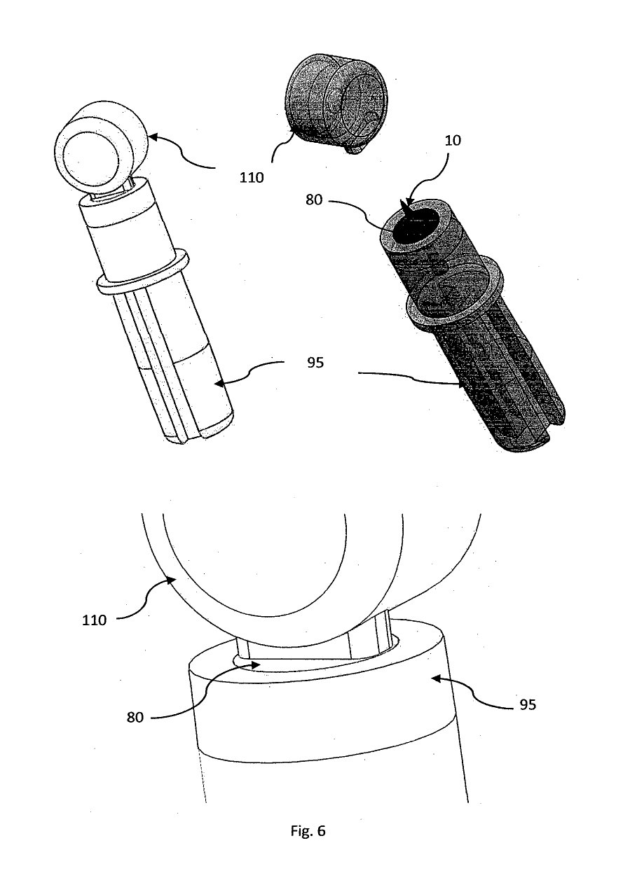

FIG. 6 shows an over-moulded skin penetration device 200 comprising a skin penetration device 10 that is over-moulded with a cap 110 and an over-moulded body 95. The over-moulded device 200 has the skin penetration device 10 moulded with a first material with sufficient toughness and strength, and the cap 110 and the over-moulded body 95 moulded with a second material which has no chemical bonding to the first material. The cap 110 is over-moulded in such a way that it is detachably adhered to the flange 80 of the skin penetration device 10. The cap 110 isolates the entire needle segment 20 from the environment to protect it from physical damages and to conserve its sterile condition till point of use.

With the full disclosure of the present invention, it is straightforward to realize several novel features which are provided by the present invention, as follows:

1. Tip disablement—the sharp tip can be disabled, i.e. bent or deformed so that the used article will not be an injury threat to the users or medical personnel, and cannot be re-used. The sharp tip can be disabled by pushing the PLancets against a hard and rigid surface, or by a mechanism incorporated within this medical device or in a lancet applicator.

2. Tip preservation—once sterilized, the article's sterile condition can be conserved reliably and cost effectively until point of use by over-moulding a detachable cap using a second polymer such as polyethylene or polypropylene onto the tips of the articles. Alternatively, the articles can be stacked in such a way that an article is capped and sealed by its adjacent neighbour. This provides saving on the packaging effort and material.

3. The over-moulding of caps using a second polymer can be further extended to over-moulding of a cap and/or an adaptor. In this case, the articles which are made separately using expensive polymer are over-moulded with soft and inexpensive polymers which do not form chemical bonding with the first material. In this case, the material cost is minimized.

4. The articles are totally made of polymers. Disposal and recycling of used articles are much easier since the melting temperature for polymers are below 350 degree Celsius. Hospitals or mobile clinics can get rid of the disposals easily by using a normal oven.

5. The articles can be made with one or two injection moulding processes. Since no stainless steel needle is needed, the production lead time and material costs are significantly reduced. The articles are the most cost efficient way for blood sampling.

The description of the present disclosure provides an accurate and exact disclosure of the preferred embodiments of the present invention. It can be understood that any further modifications that fall within the inventive spirit of the present invention shall be considered within the scope of the present invention.

1. A surgically invasive pointed article, the article comprising a base surface, two body segments which are unitarily manufactured using a first material, the first body segment being a needle segment which extends from the base surface, and the second body segment being a body portion for handling purpose, wherein the needle segment can be deformed such that the surgically invasive function is disabled to prevent accidental injuries and re-use.

2. The surgically invasive pointed article in claim 1, wherein the first material is of high toughness and strength such as polycarbonate and polyetherimide.

3. The surgically invasive pointed article in claim 1, wherein a second material is over-moulded onto the article such that a cap is formed and is detachably adhered to the base surface of claim 1, and that the entire first body segment is encapsulated to protect the pointed end from damage, and once sterilized, to conserve the sterile conditions till point of use.

4. The surgically invasive pointed article in claim 3, wherein a second material is over-moulded onto the article to form an over-moulded body such that the cap encapsulates the entire first body segment and detachably adheres to the base surface, and the over-moulded body covers a portion of the second body segment to protect the pointed end from damage, and once sterilized, to conserve the sterile conditions till point of use.

5. The surgically invasive pointed article in claim 3, wherein the second material used for over-moulding is a soft and inexpensive commodity polymer such as silicone, polyethylene or polypropylene.

6. The method of obtaining blood sample using the surgically invasive pointed article in claim 1, the method comprising lancing a body site with the article and subsequently disabling the protruding pointed end to prevent injuries and re-use.

7. A surgically invasive pointed article comprising
a. injection moulding of a first material to form a first part further comprising
i. a base surface,
ii. two body segments, the first body segment being a needle segment which extends from the base surface, and the second body segment being a body portion for handling purpose,
wherein the needle segment can be deformed such that the surgically invasive function is disabled to prevent accidental injuries and re-use;
b. over-moulding a second material to form a second part further comprising a cap which is detachably adhered to the base surface and that the entire first body segment is encapsulated to protect the pointed end from damage, and once sterilized, to conserve the sterile conditions till point of use.

8. The surgically invasive pointed article in claim 7, wherein the first material is of high toughness and strength such as polycarbonate and polyetherimide.

9. The surgically invasive pointed article in claims 7, wherein the second material used for over-moulding is a soft and inexpensive commodity polymer such as silicone, polyethylene or polypropylene.

10. A method for manufacturing a surgically invasive pointed article, comprising
a. injection moulding of a first material to form a first part further comprising forming
i. a base surface,
ii. two body segments, the first body segment being a needle segment which extends from the base surface, and the second body segment being a body portion for handling purpose,
wherein the needle segment can be deformed such that the surgically invasive function is disabled to prevent accidental injuries and re-use;
c. over-moulding a second material to form a second part further comprising a cap which is detachably adhered to the base surface and that the entire first body segment is encapsulated to protect the pointed end from damage, and once sterilized, to conserve the sterile conditions till point of use.

11. The surgically invasive pointed article in claim 10, wherein the first material is of high toughness and strength such as polycarbonate and polyetherimide.

12. The surgically invasive pointed article in claim 10, wherein the second material used for over-moulding is a soft and inexpensive commodity polymer such as silicone, polyethylene or polypropylene.
